(19) United States
(12) Patent Application Publication
(10) Pub. No.: US 2024/0157096 A1
(43) Pub. Date: May 16, 2024

(54) CATHETER SYSTEM HAVING A GUIDEWIRE

(71) Applicant: Becton, Dickinson and Company, Franklin Lakes, NJ (US)

(72) Inventors: S. Ray Isaacson, Layton, UT (US); Weston F. Harding, Lehi, UT (US); Ralph L. Sonderegger, Farmington, UT (US); Jonathan Karl Burkholz, Salt Lake City, UT (US); Edwin Jeyaseelan A., Tamil Nadu (IN); Mukilan Balamurugan, Tamil Nadu (IN)

(21) Appl. No.: 18/502,999

(22) Filed: Nov. 6, 2023

Related U.S. Application Data
(60) Provisional application No. 63/425,896, filed on Nov. 16, 2022.

(51) Int. Cl. A61M 25/09; A61M 25/06

(52) U.S. Cl. CPC A61M 25/09041; A61M 25/0618; A61M 2202/0007

(57) ABSTRACT

A catheter system may include a catheter assembly and a needle assembly. The catheter assembly may include a catheter adapter, which may include a distal end, a proximal end, a lumen, and a side port. The catheter assembly may include a catheter extending from the distal end of the catheter adapter and an extension tube. A distal end of the extension tube may be integrated with the side port of the catheter adapter. The needle assembly may include a needle safety element, which may be disposed within the safety housing or the catheter adapter. The needle assembly may include a guidewire advancement device coupled to the safety housing or the catheter adapter, and an introducer needle having a proximal end coupled to the guidewire advancement device.

### RELATED APPLICATIONS

This application claims the benefit of U.S. Provisional Patent Application No. 63/425,896, which was filed on Nov. 16, 2022, which is incorporated herein in its entirety.

### BACKGROUND

Arterial catheterization is a vital procedure that is used ubiquitously in the hospital setting, both in critically injured and perioperative patients. It is estimated that more than eight million arterial catheters are placed yearly in the United States. Arterial catheters can accurately measure blood pressure as well as heart rate and pulse contour to allow for immediate recognition of aberrant hemodynamic events and initiation of appropriate treatment. Arterial catheters also provide samples for blood gas analysis without the morbidity associated with repeat arterial puncture. However, use of current arterial catheters can result in significant blood leakage during insertion into an artery of a patient, which can endanger a user. Moreover, current arterial catheters may be difficult to use with a probe or a catheter advancement device.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system, as well as related devices and methods. In some embodiments, the catheter system may be configured for blood sampling, such as, for example, arterial blood sampling. In some embodiments, the catheter system may be configured for blood pressure monitoring and/or blood gas sampling. Importantly, in some embodiments, the catheter system may provide near-patient access for more accurate hemodynamic measurements and improved delivery of an instrument, such as a secondary catheter and/or a probe, into a blood vessel, which may include an artery or a vein. In some embodiments, the catheter system may include a guidewire for improved catheter insertion success. In some embodiments, the catheter system may reduce blood exposure when inserted the catheter into an artery of a patient.

In some embodiments, the catheter system may include a catheter assembly. In some embodiments, the catheter assembly may include a catheter adapter, which may include a distal end, a proximal end, a lumen extending through the distal end of the catheter adapter and the proximal end of the catheter adapter. In some embodiments, the catheter adapter may also include a side port between the distal end of the catheter adapter and the proximal end of the catheter adapter and in fluid communication with the lumen. In some embodiments, the catheter assembly may include the catheter extending from the distal end of the catheter adapter.

In some embodiments, the catheter assembly may include an extension tube, which may include a distal end and a proximal end. In some embodiments, the distal end of the extension tube may be integrated with the side port of the catheter adapter. In some embodiments, the extension tube may be a first extension tube. In some embodiments, the catheter system may include a second extension tube, which may include a distal end and a proximal end. In some embodiments, the catheter system may include an access connector, which may be configured to provide near-patient access. In some embodiments, the access connector may include a distal port, a proximal port, and a side port between the distal port and the proximal port. In some embodiments, the distal port and the proximal port may be aligned with a longitudinal axis of the access connector. In some embodiments, the side port may be angled with respect to the longitudinal axis of the access connector. In some embodiments, the proximal end of the first extension tube may be integrated with the distal port of the access connector.

In some embodiments, the distal end of the second extension tube may be integrated with the side port of the access connector. In some embodiments, the first extension tube may be shorter than the second extension tube such that the first extension tube facilitates advancement of a secondary catheter and/or probe through the first extension tube. In some embodiments, the longitudinal axis of the access connector, the first extension tube, and the side port may be configured to align to form a straight path, which may facilitate advancement of the secondary catheter and/or the probe within the catheter system. In some embodiments, the first extension tube may be rigid or semi-rigid, which may facilitate advancement of the secondary catheter and/or the sensor therethrough.

In some embodiments, the proximal port may include a female luer, which may facilitate coupling of a catheter advancement device to the access connector. In some embodiments, the proximal port may include another suitable connector. In some embodiments, the catheter advancement device may be coupled to the proximal port. In some embodiments, the catheter advancement device may include the PIVO™ Needle-Free Blood Collection Device, available from Becton, Dickinson & Company of Franklin Lakes, New Jersey, or another suitable catheter advancement device. In some embodiments, the catheter advancement device may include a blood sampling device.

In some embodiments, the catheter system may include a needle assembly, which may include one or more of the following: a safety housing, a guidewire advancement device, and an introducer needle. An active safety mechanism (e.g., pushing a button) requires user activation of the active safety mechanism, whereas a passive safety mechanism is activated automatically during normal use of the catheter system (e.g., withdrawal of the introducer needle). In some embodiments, the catheter system may include a needle safety element configured to shield a sharp distal tip of the introducer needle. In some embodiments, the needle safety element may include a V-clip needle shield, a crossing-arm clip, or another suitable spring clip or another passive safety mechanism configured to shield the introducer needle. In some embodiments, the needle safety element may be disposed within the safety housing or within the catheter adapter. In some embodiments, the needle safety element disposed within the safety housing may protect a user from blood splatter that may exit the sharp distal tip of the introducer needle and may also shield the user from the sharp distal tip. In some embodiments, the safety housing may include a distal end and a proximal end. In some embodiments, the distal end of the safety housing may be coupled to the proximal end of the catheter adapter.

In some embodiments, the guidewire advancement device may include a distal end and a proximal end. In some embodiments, the distal end of the guidewire advancement device may be coupled to the proximal end of the safety housing. In some embodiments without the safety housing, the distal end of the guidewire advancement device may be coupled to the proximal end of the catheter adapter. In some embodiments, the introducer needle may include a proximal end coupled to the guidewire advancement device. In some embodiments, the introducer needle may extend through the catheter. In some embodiments, removal of the guidewire advancement device from the catheter system may be configured to withdraw the guidewire and the introducer needle from the catheter system. In some embodiments, the guidewire may be configured to remain proud of the introducer needle when the guidewire and the introducer needle are withdrawn from the catheter system.

In some embodiments, the guidewire advancement device may include a slot and an advancement tab moveable linearly along the slot. In some embodiments, the advancement tab may be configured to advance the guidewire distally and/or withdraw the guidewire proximally. In some embodiments, a longitudinal axis of the guidewire advancement device may be aligned with a longitudinal axis of the catheter assembly. In some embodiments, the guidewire advancement device may include a rack and a pinion. In some embodiments, the advancement tab may include the rack. In some embodiments, the guidewire advancement device may include a rotary element configured to advance the guidewire distally and/or retract the guidewire proximally.

In some embodiments, the introducer needle may include the sharp distal tip, a proximal end, and a lumen extending through the sharp distal tip and the proximal end. In some embodiments, the guidewire may be disposed within the lumen of the introducer needle and/or may be configured to move through the proximal end of the introducer needle to advance distally. In some embodiments, the introducer needle may include an external groove. In some embodiments, the guidewire may be disposed within the external groove and may be configured to move through the external groove to advance distally and/or be withdrawn proximally.

In some embodiments, the safety housing may include an open space proximate and proximal to the V-clip needle shield. In some embodiments, the guidewire may be configured to bend within the open space when the V-clip needle shield fires. In some embodiments, the sharp distal tip may include a bevel facing upward, and the V-clip needle shield may be configured to fire side to side. In some embodiments, the sharp distal tip may include the bevel facing upward, and the V-clip needle shield may be configured to fire in an upward direction or a downward direction.

In some embodiments, the introducer needle may include an aperture disposed between the sharp distal tip and the proximal end of the introducer needle. In some embodiments, the guidewire may be configured to extend through the extension tube, the side port, and the aperture and out the sharp distal tip of the introducer needle.

In some embodiments, the proximal end of the second extension tube may be integrated with an adapter. In some embodiments, the adapter may be coupled to a needleless connector, which may reduce a risk of bacterial contamination. In some embodiments, a proximal end of the adapter may include a single port or a dual port.

In some embodiments, a method may include inserting a catheter system into a blood vessel of a patient. In some embodiments, the blood vessel may include an artery. In some embodiments, the catheter system may be inserted into the blood vessel by puncturing the skin and the blood vessel with the introducer needle and positioning the catheter within the blood vessel. In some embodiments, the method may include advancing the guidewire distally via the guidewire advancement device after inserting the catheter system into the blood vessel. In some embodiments, after advancing the guidewire distally, the method may include removing the guidewire advancement device from the catheter system. In some embodiments, removal of the guidewire advancement device from the catheter system withdraws the guidewire and the introducer needle from the catheter system. In some embodiments, the guidewire may remain proud of the introducer needle when the guidewire and the introducer needle are withdrawn from the catheter system.

In some embodiments, the method may include after removing the guidewire advancement device from the catheter system, coupling the catheter advancement device to the proximal port and advancing a secondary catheter into the catheter assembly via the catheter advancement device. In some embodiments, the method may include after removing the guidewire advancement device from the catheter system, coupling a probe advancement device to the proximal port and advancing a probe of the probe advancement device into the catheter assembly. In some embodiments, the probe may include one or more sensors configured to measure one or more properties of blood.

According to another set of embodiments, a catheter system may include a catheter adapter, which may include a distal end, a proximal end, a lumen extending through the distal end of the catheter adapter and the proximal end of the catheter adapter. In some embodiments, the catheter system may include a catheter extending from the distal end of the catheter adapter. In some embodiments, the catheter system may include a guidewire advancement device coupled to the catheter adapter. In some embodiments, the guidewire advancement device may include a casing, a guidewire at least partially disposed within the casing, and a roller wheel element extending through the casing and contacting the guidewire. In some embodiments, in response to rotation of the roller wheel element in a proximal direction, the guidewire may be configured to advance distally. In some embodiments, the catheter system may include an introducer needle, which may include a proximal end and a sharp distal tip. In some embodiments, the proximal end of the introducer needle may be secured within the guidewire advancement device. In some embodiments, the introducer needle may extend through the catheter.

In some embodiments, the catheter system may include a platform disposed within the casing and below the roller wheel element. In some embodiments, the platform may include a linear groove. In some embodiments, the guidewire may be disposed within the linear groove, which may facilitate guidance of the guidewire during distal advancement of the guidewire. In some embodiments, the catheter system may include opposing roller wheel support groves disposed within the casing. In some embodiments, the roller wheel element comprises opposing pins configured to rotate within the opposing support grooves.

In some embodiments, a distal end of the casing may include a male leer fitting, which may facilitate coupling to the catheter adapter and/or a safety housing. In some embodiments, the catheter system may include a septum and a septum actuator disposed within the lumen of the catheter adapter. In some embodiments, the septum may help stop blood flow through the catheter adapter when the catheter adapter is not engaged with an external connector or extension set. In some embodiments, a fluid path through the catheter adapter may be closed unless the external connector is engaged with the catheter adapter such that the septum actuator opens the septum.

According to another set of embodiments, a guidewire advancement device coupled to the catheter adapter may include a casing, a slot within the casing, a guidewire, and an advancement tab extending through the slot and configured to move along the slot to advance the guidewire distally.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, illustrating an example linear guidewire advancement device, according to some embodiments;

FIG. 1B is a cross-sectional view of the catheter system, illustrating an example advancement tab in an example proximal position, according to some embodiments;

FIG. 1C is a cross-sectional view of the catheter system, illustrating the advancement tab in an example distal position, according to some embodiments;

FIG. 2A is an upper perspective view of the catheter system, illustrating an example rotary guidewire advancement device, according to some embodiments;

FIG. 2B is a cross-sectional view of the catheter system, illustrating an example rotary element in a first position, according to some embodiments;

FIG. 2C is a cross-sectional view of the catheter system, illustrating the rotary element rotated to a second position, according to some embodiments;

FIG. 2D provides an exploded rear view of the rotary element in isolation, according to some embodiments.

FIG. 3A is an upper perspective view of an example introducer needle having a groove, according to some embodiments;

FIG. 3B is a cross-sectional view of the introducer needle, according to some embodiments;

FIG. 4A is an upper perspective view of the introducer needle having an example aperture and a guidewire extending through the aperture, according to some embodiments;

FIG. 4B is a partial cutaway view of the catheter system, illustrating the introducer needle having the aperture, according to some embodiments;

FIG. 4C is a cross-sectional view of an example adapter, according to some embodiments;

FIG. 5A is an upper perspective view of an example V-clip needle shield, according to some embodiments;

FIG. 5B is a cross-sectional view of the V-clip needle shield separating from an example catheter adapter groove, according to some embodiments;

FIG. 5C is a cross-sectional view of the catheter system, illustrating the introducer needle withdrawn proximally into an example safety housing prior to separation of the safety housing from an example catheter adapter, according to some embodiments;

FIG. 6A is a partial cut-away view of the catheter system, illustrating an example capture mechanism in which the introducer needle is exposed from a distal end of an example inner housing, according to some embodiments;

FIG. 6B is a partial-cutaway view of the catheter system, illustrating the capture mechanism with the introducer needle retracted within the inner housing, according to some embodiments;

FIG. 6C is a partial-cutaway view of the catheter system, illustrating the capture mechanism being removed from the catheter adapter, according to some embodiments;

FIG. 7 is an upper perspective view of an example spring clip with the introducer needle shielded therein, according to some embodiments;

FIG. 8A is a cross-sectional view of an example pinion and an example advancement tab having an example rack, illustrating the advancement tab in a first, proximal position, according to some embodiments; and

FIG. 8B is a cross-sectional view of the pinion and the advancement tab having the example rack, illustrating the advancement tab in an example second, distal position, according to some embodiments;

FIG. 9A is a side view of another example catheter system, according to some embodiments;

FIG. 9B is a cross-sectional view of the catheter system of FIG. 9, according to some embodiments;

FIG. 9C is another cross-sectional view of the catheter system of FIG. 9 transverse to the cross-sectional view of FIG. 9B, according to some embodiments;

FIG. 9D is an upper perspective view of the catheter system of FIG. 9, illustrating an example casing in an open position, according to some embodiments;

FIG. 9E is an upper perspective view of an example septum actuator, according to some embodiments;

FIG. 9F is an upper perspective view of an example septum, according to some embodiments;

FIG. 9G is an upper perspective view of an example roller wheel element disposed within example opposing support grooves, according to some embodiments;

FIG. 9H is an upper perspective view of the roller wheel element disposed within the opposing support grooves, according to some embodiments;

FIG. 10A is an upper perspective view of another catheter system, illustrating an example advancement tab in an example proximal position, according to some embodiments;

FIG. 10B is an upper perspective view of the advancement tab coupled to an example guidewire, according to some embodiments; and

FIG. 10C is an upper perspective view of the catheter system of FIG. 10A, illustrating the advancement tab in an example distal position, according to some embodiments.

### DESCRIPTION OF EMBODIMENTS

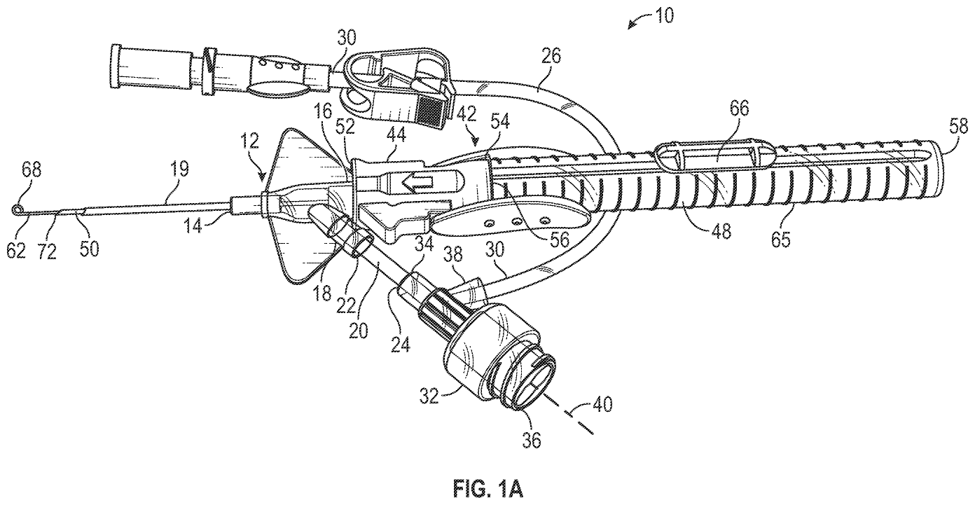

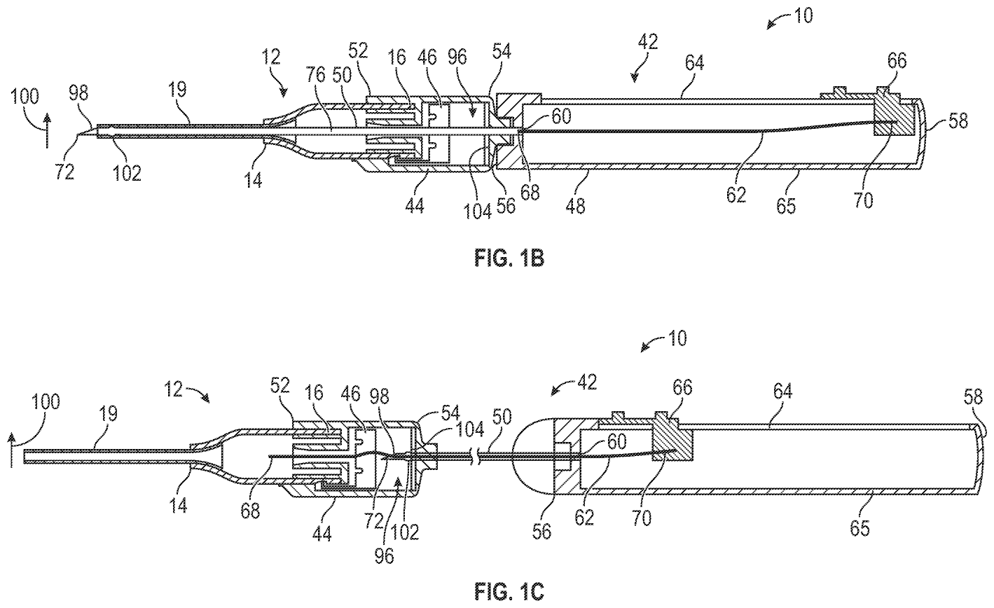

Referring now to FIGS. 1A-1C, a catheter system 10 is illustrated, according to some embodiments. In some embodiments, the catheter system 10 may be configured for blood sampling, such as, for example, arterial blood sampling. In some embodiments, the catheter system 10 may also be configured for blood pressure monitoring and/or blood gas sampling. Importantly, in some embodiments, the catheter system 10 may provide near-patient access for more accurate hemodynamic measurements and improved delivery of an instrument, such as a secondary catheter and/or a probe, into a blood vessel, which may include an artery or a vein. In some embodiments, the secondary catheter may include another catheter configured to extend through the catheter 19 to extend a life of the catheter 19, and the secondary catheter may be used for blood withdrawal and/or infusion.

In some embodiments, the catheter system 10 or a catheter system of one or more of FIGS. 1-8 may be referred to as an “integrated” catheter system, meaning that the catheter system includes extension tubing (e.g., an extension set) that provides a fluid pathway to the catheter. In some embodiments, the catheter system 10 may be similar the NEXIVA™ Closed IV Catheter System, the NEXIVA™ DIFFUSICS™ Closed IV Catheter System, or the PEGASUS™ Safety Closed IV Catheter System (all available from Becton Dickinson & Company of Franklin Lakes, New Jersey) or another suitable integrated catheter system in terms of one or more components and/or operation.

In some embodiments, the catheter system 10 may include an arterial catheter system configured for insertion into an artery. In these embodiments, the catheter system 10 may include significant improvements to existing arterial catheter systems by dramatically reducing blood exposure and infection risk and/or providing the user with improved artery access confirmation, improving an overall experience of a patient. Some existing arterial catheter systems, such as, for example, the Teleflex ARROW ° Integrated Arterial Catheter, may not provide effective artery blood control, which may result in placement procedures with significant blood exposure risk, infection risk, clean-up costs, and poor patient experience. The Teleflex ARROW ° Integrated Arterial Catheter includes a non-rigid, slotted tube out of which significant amounts of blood may leak, endangering the user.

The catheter system 10 may include one or more of the following, which may provide advantages over the prior art: arterial blood sampling with reduced blood exposure; blood pressure monitoring; blood gas sampling; near-patient access for use of a secondary catheter and/or a probe; blood control configured to operate under arterial pressure; a guidewire; and magnetic introducer needle guidance technology.

As illustrated in FIGS. 1A-1C, in some embodiments, the catheter system 10 may include a catheter assembly which may include a catheter adapter 12, which may include a distal end 14, a proximal end 16, a lumen extending through the distal end 14 of the catheter adapter 12 and the proximal end 16 of the catheter adapter 12. In some embodiments, the catheter adapter 12 may also include a side port 18 between the distal end 14 of the catheter adapter 12 and the proximal end 16 of the catheter adapter 12 and in fluid communication with the lumen. In some embodiments, the catheter assembly may include a catheter 19 extending from the distal end 14 of the catheter adapter 12. In some embodiments, the catheter 19 may include an arterial catheter, a peripherally-inserted central catheter, a midline catheter, a peripheral intravenous catheter, or another suitable catheter.

In some embodiments, the catheter assembly may include an extension tube 20, which may include a distal end 22 and a proximal end 24. In some embodiments, the distal end 22 of the extension tube 20 may be integrated with the side port 18 of the catheter adapter 12, which may reduce a risk of fluid exposure to the user. In further detail, in some embodiments, the distal end 22 of the extension tube 20 may be permanently or non-removably coupled to the side port 18 of the catheter adapter 12, such as, for example, via adhesive, bonding, a non-luger coupling, or another suitable permanent or non-removable coupling.

In some embodiments, the extension tube 20 may be a first extension tube. In some embodiments, the catheter system 10 may include a second extension tube 26, which may include a distal end 28 and a proximal end 30. In some embodiments, the catheter system 10 may include an access connector 32, which may be configured to provide near-patient access for one or more of blood sampling, a probe, or a secondary catheter. In some embodiments, the access connector 32 may include a distal port 34, a proximal port 36, and a side port 38 between the distal port 34 and the proximal port 36. In some embodiments, the distal port 34 and the proximal port 36 may be aligned with a longitudinal axis 40 of the access connector 32. In some embodiments, the side port 38 may be angled with respect to the longitudinal axis 40 of the access connector 32. In some embodiments, the proximal end 24 of the extension tube 20 may be integrated with the distal port 34 of the access connector 32, which may reduce a risk of fluid exposure to the user. In further detail, in some embodiments, the proximal end 24 of the extension tube 20 may be permanently or non-removably coupled to the distal port of the access connector, such as, for example, via adhesive, bonding, a non-luer coupling, or another suitable permanent or non-removable coupling.

In some embodiments, the distal end 28 of the second extension tube 26 may be integrated with the side port 38 of the access connector 32, which may reduce a risk of fluid exposure to the user. In further detail, in some embodiments, the distal end 28 of the second extension tube 26 may be permanently or non-removably coupled to the side port 38 of the access connector 32, such as, for example, via adhesive, bonding, a non-luer coupling, or another suitable permanent or non-removable coupling. In some embodiments, the extension tube 20 may be short to facilitate near-patient access. In some embodiments, the extension tube 20 may be shorter than the second extension tube 26, such that the extension tube 20 facilitates advancement of a secondary catheter and/or probe through the extension tube 20 and the proximal port 36. In some embodiments, the longitudinal axis 40 of the access connector 32, the extension tube 20, and the side port 18 may be configured to align to form the straight path, which may facilitate advancement of the secondary catheter and/or the probe within the catheter system 10. In some embodiments, the extension tube 20 may be rigid or semi-rigid, which may facilitate advancement of the secondary catheter and/or the probe therethrough.

In some embodiments, the proximal port 36 may include a female luer, which may facilitate coupling of a catheter advancement device to the access connector 32. In some embodiments, the proximal port 36 may include another suitable connector. In some embodiments, the catheter advancement device may be coupled to the proximal port 36. In some embodiments, the catheter advancement device may include the PIVO™ Needle-Free Blood Collection Device, available from Becton, Dickinson & Company of Franklin Lakes, New Jersey, or another suitable catheter advancement device. In some embodiments, the catheter advancement device may include a blood sampling device.

In some embodiments, the catheter system 10 may include a needle assembly 42, which may include one or more of the following: a safety housing 44, a V-clip needle shield 46 disposed within the safety housing 44, a guidewire advancement device 48, and an introducer needle 50. In some embodiments, the safety housing 44 may include a distal end 52 and a proximal end 54. In some embodiments, the distal end 52 of the safety housing 44 may be coupled to the proximal end 16 of the catheter adapter 12.

In some embodiments, the guidewire advancement device 48 may include a distal end 56 and a proximal end 58. In some embodiments, the distal end 56 of the guidewire advancement device 48 may be removably coupled to the proximal end 54 of the safety housing 44. In some embodiments, the introducer needle 50 may include a proximal end 60 coupled to the guidewire advancement device 48 such that the proximal end 60 is secured within the guidewire advancement device 48. In some embodiments, the introducer needle 50 may extend through the catheter 19 to facilitate insertion of the catheter 19 into the blood vessel. In some embodiments, the guidewire advancement device 48 may be configured to advance a guidewire 62 distally and/or withdraw the guidewire 62 proximally.

In some embodiments, removal of the guidewire advancement device 48 from the catheter system 10 may be configured to withdraw the guidewire 62 and the introducer needle 50 from the catheter system 10, as illustrated, for example, in FIG. 1C. In further detail, in some embodiments, the guidewire 62 and the introducer needle 50 may be withdrawn together in the proximal direction. In some embodiments, the guidewire 62 may be configured to remain proud of the introducer needle 50 when the guidewire 62 and the introducer needle 50 are withdrawn from the catheter system 10. This may reduce a risk of shearing of the guidewire 62.

In some embodiments, the guidewire advancement device 48 may include a slot 64 and an advancement tab 66 moveable linearly along the slot 64. In some embodiments, the slot 64 may be disposed within a housing 65. In some embodiments, the advancement tab 66 may be configured to advance the guidewire 62 distally and/or withdraw the guidewire 62 proximally. In some embodiments, a longitudinal axis of the guidewire advancement device 48 may be aligned with a longitudinal axis of the catheter assembly.

In some embodiments, the guidewire 62 may facilitate advancement of the catheter 19 within the blood vessel and/or removal of a thrombus or occlusion. In some embodiments, the guidewire 62 may include a distal end 68 and a proximal end 70. In some embodiments, the proximal end 70 may be coupled to the advancement tab 66, which may be configured to slide along the slot 64 by a hand of the user. In some embodiments, the distal end 68 of the guidewire 62 may be disposed within the guidewire advancement device 48 and/or the introducer needle 50 when the advancement tab 66 is in a proximal position, as illustrated, for example, in FIG. 1B. In these and other embodiments, the guidewire 62 placed within the introducer needle 50 may facilitate guidance of the guidewire 62 as it is advanced distally. In some embodiments, the advancement tab 66 may be at a proximal end of the slot 64 when the advancement tab 66 is in the proximal position and/or at a distal end of the slot 64 when the advancement tab 66 is in a distal position. In some embodiments, the advancement tab 66 may be slid from the proximal position to the distal position to move the distal end 68 of the guidewire 62 beyond a distal tip of the catheter 19 and into the blood vessel.

In some embodiments, the introducer needle 50 may include a sharp distal tip 72, the proximal end 60, and a lumen 76 extending through the sharp distal tip 72 and the proximal end 60. In some embodiments, the guidewire 62 may be disposed within the lumen 76 of the introducer needle 50 and/or may be configured to move through the proximal end 60 of the introducer needle 50 to advance distally. In these embodiments, the proximal end 60 of the introducer needle 50 may be generally cylindrical and/or not crimped, which may facilitate movement of the guidewire 62 therethrough.

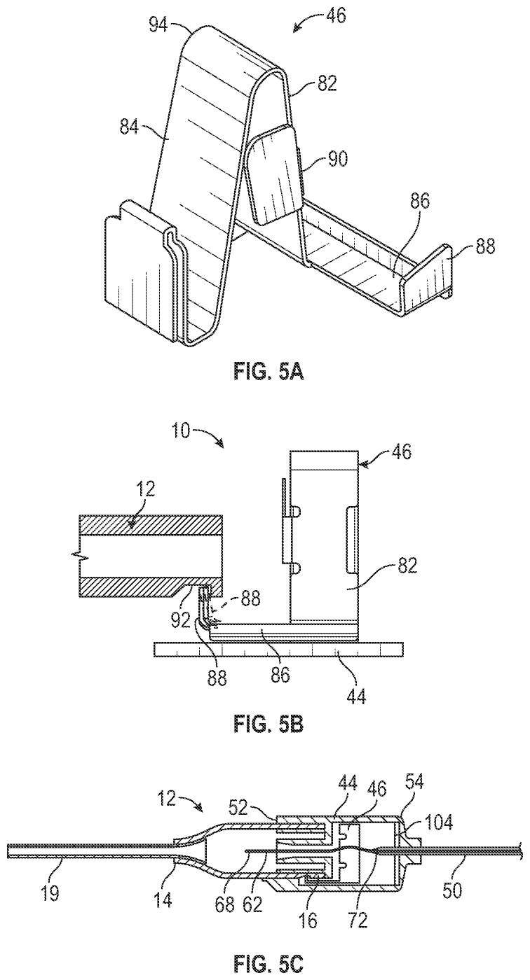

Referring now to FIGS. 5A-5B, the V-clip needle shield 46 may include a first arm 82 and a second arm 84. In some embodiments, the first arm 82 may include an extension 86 forming a pawl 88 at the end of the extension 86. In some embodiments, the first arm 82 and/or the second arm 84 may also include a needle tip shield flap 90 capable of halting the advancement of the sharp distal tip 72 after the V-clip needle shield 46 is engaged. In some embodiments, the needle tip shield flap 90 may be used to prevent the reemergence of the sharp distal tip 72 from the safety housing 44 after the introducer needle 50 has been shielded by the safety housing 44.

In some embodiments, the pawl 88 may be formed of a metal that is bent at a primarily 90-degree bend from the extension 86. However, in some embodiments, the pawl 88 may be bent at any other desired angle. In some embodiments, the pawl 88 may be bent in the range of about 30 degrees to about 180 degrees with respect to the extension 86. In some embodiments, the pawl 88 may be bent in any direction that allows the pawl 88 to engage the catheter adapter 12. In some embodiments, the pawl 88 on the V-clip needle shield 46 may serve to engage a crescent-shaped groove 92 on the catheter adapter 12 (see, for example, FIG. 5B). In some embodiments, the pawl 88 may engage the groove 92 to prevent the safety housing 44 from separating from the catheter adapter 12 until the introducer needle 50 is withdrawn far enough to activate the V-clip needle shield 46. FIG. 5B illustrates a pawl 88 being separated from the groove 92, according to some embodiments.

In some embodiments, when the V-clip needle shield 46 is activated (fires), the first arm 82 will, under the spring force of an elbow 94, separate from the second arm 84. In some embodiments, as the first arm 82 separates from the second arm 84, the pawl 88 will move from a first position to a second position. In some embodiments, when the pawl 88 is in its first position, it is engaged with the groove 92 on the catheter adapter 12. In some embodiments, after the pawl 88 moves from its first position to its second position, the pawl 88 moves from engagement with the groove 92 to a position that is out of engagement with the groove 92.

In some embodiments, in order to prevent premature disengagement of the pawl 88 from the groove 92, the pawl 88 must be of sufficient strength to avoid any bending, movement, shifting, or other action that would cause the V-clip needle shield 46 to separate from the catheter adapter 12 before the introducer needle 50 has advanced far enough to activate or engage the V-clip needle shield 46. In some embodiments, the V-clip needle shield may include any suitable V-clip needle shield or may be further described in U.S. Pat. No. 9,220,871, filed Nov. 20, 2007, entitled “NEEDLE SHIELDING PAWL STRUCTURES,” which is hereby incorporated by reference in its entirety.

Referring back to FIGS. 1A-1C, in some embodiments, the safety housing 44 may include an open space 96 proximate and proximal to the V-clip needle shield 46. In some embodiments, the open space 96 may be provided in an axial direction or in a direction of a longitudinal axis of the catheter system 10. In some embodiments, the guidewire 62 may be configured to bend within the open space 96 when the V-clip needle shield 46 fires, as illustrated, for example, in FIG. 1C. In some embodiments, as illustrated in FIGS. 1B-1C, for example, a width of the open space 96 may be equal to or greater than a width of the V-clip needle shield 46, which may allow the V-clip needle shield 46 to properly fire when the guidewire 62 is extended distally beyond the sharp distal tip 72. In some embodiments, the sharp distal tip 72 may include a bevel 98 facing toward a top of the catheter system 10 or in an upward direction 100 (see, for example, FIGS. 1B-1C), and the V-clip needle shield 46 may be configured to fire side to side, perpendicular to the upward direction 100. When the bevel 98 is facing in the upward direction 100, the catheter system 10 is in an insertion position ready for insertion into the patient.

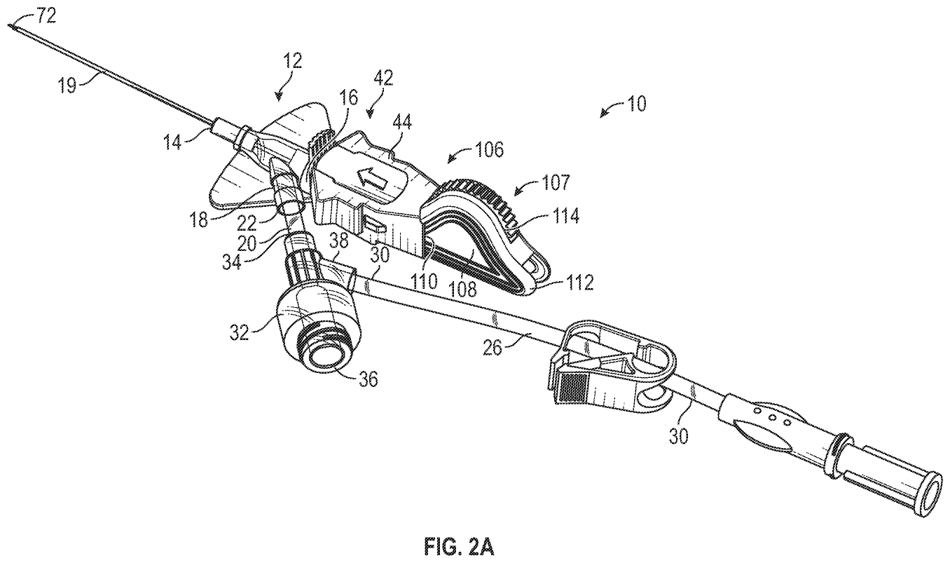

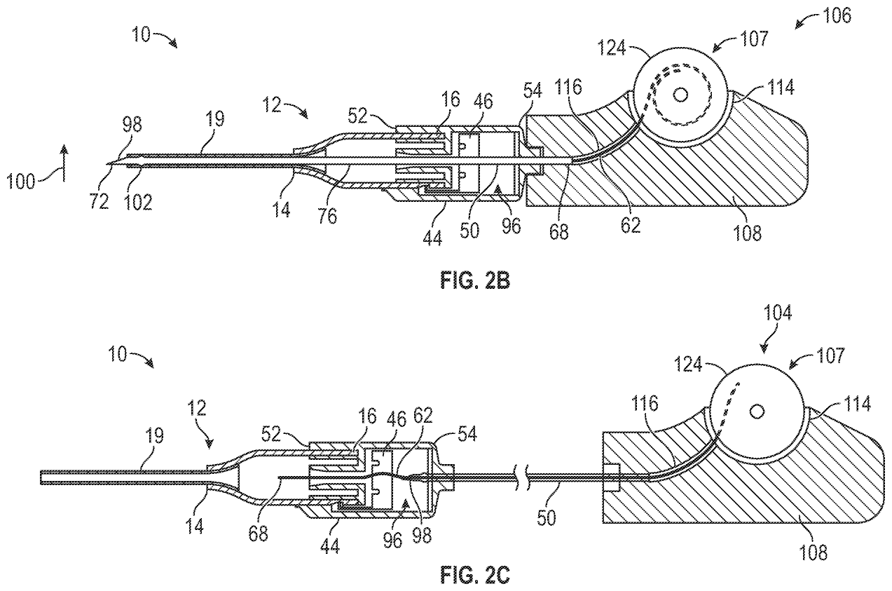

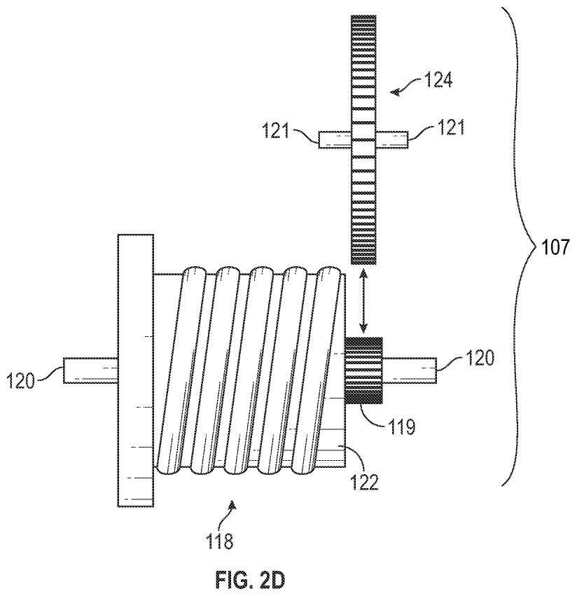

Referring now to FIG. 5C, in some embodiments, the sharp distal tip 72 may include the bevel 98 facing in the upward direction (directly out of the page), and the V-clip needle shield 46 may be configured to fire in the upward direction or a downward direction opposite the upward direction. The cross-sectional view in FIG. 5C is a top view and illustrates a bottom of the catheter system, configured to be closest to the patient, according to some embodiments. Thus, in some embodiments, the open space 96 may be provided perpendicular to the axial direction or perpendicular to the direction of the longitudinal axis of the catheter system 10. In some embodiments, the introducer needle 50 may include the needle bump 102 (see, for example, FIG. 2B), which may prevent the introducer needle 50 from being proximally removed from the safety housing 44.

Referring back to FIGS. 1A-1C, in some embodiments, the proximal end 54 of the safety housing 44 may be removably coupled to the guidewire advancement device 48. In some embodiments, the introducer needle 50 may include a needle bump 102, which may catch on a washer 104 having a hole with a smaller diameter than the needle bump 102. Thus, in some embodiments, when the guidewire advancement device 48 is moved proximally to retract the introducer needle 50 in the proximal direction, the needle bump 102 will not pass the hole, and the sharp distal tip 72 may be retained within the safety housing 44, preventing an accidental needle stick. In some embodiments, the distal end 52 of the safety housing 44 may be removably coupled to the proximal end 16, and when the sharp distal tip 72 is shielded within the safety housing 44, the distal end 52 of the safety housing 44 may be uncoupled from the proximal end 16.

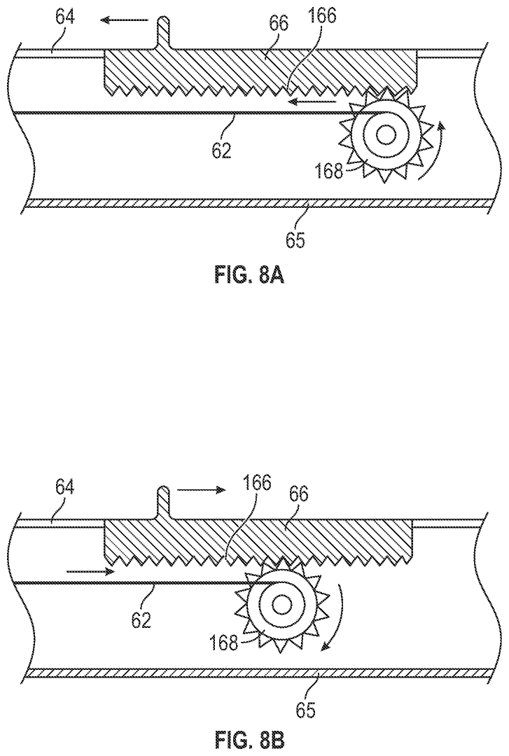

In some embodiments, the guidewire 62 may be advanced distally via a particular guidewire advancement device, such as, for example, the guidewire advancement device 48 of FIG. 1, a guidewire advancement device 106 of FIG. 2, a guidewire advancement device 165 of FIG. 8, or another suitable guidewire advancement device. In some embodiments, use of an integrated catheter system may facilitate separation of a particular guidewire advancement device with a fluid pathway or blood collection pathway through the extension tube 20. In some embodiments, the particular guidewire advancement device may be removed from the catheter system 10 after use. In some embodiments, removal of the particular guidewire advancement device from the catheter system 10 may be configured to withdraw or remove the guidewire 62 and the introducer needle from the catheter system 10, or more specifically the catheter assembly. In some embodiments, the guidewire 62 may remain proud of the introducer needle 50 when the guidewire 62 and the introducer needle 50 are withdrawn from the catheter system 10, which may prevent shearing.

In some embodiments, after removing the particular guidewire advancement device from the catheter system 10, the catheter advancement device may be coupled to the proximal port 36 and a secondary catheter may be advanced into the catheter assembly via the catheter advancement device. In some embodiments, after removing the particular guidewire advancement device from the catheter system 10, a probe advancement device may be coupled to the proximal port and a probe of the probe advancement device may be advanced into the catheter assembly. In some embodiments, the probe may include one or more sensors configured to measure one or more properties of blood.

Referring now to FIGS. 2A-2D, in some embodiments, the guidewire advancement device 106 may include a rotary element 107 configured to advance the guidewire 62 distally and/or retract the guidewire 62 proximally. In some embodiments, the guidewire advancement device 106 may be similar or identical to the guidewire advancement device 48 of FIGS. 1A-1C in terms of one or more features and/or operation.

In some embodiments, the guidewire advancement device 106 may include a housing 108 having a distal end 110 and a proximal end 112. In some embodiments, the guidewire advancement device 106 may include a rotary element 107 that enables the guidewire 62 to be advanced in a distal direction through the catheter assembly and/or subsequently withdrawn in a proximal direction. In some embodiments, a compartment 114 may be formed within the guidewire advancement device 106 and may house the rotary element 107. In some embodiments, a guidewire channel 116 may extend distally from the compartment 114 and through the distal end 110 of the housing 108.

As illustrated in FIGS. 2B-2C, in some embodiments, the rotary element 107 may include an advancement wheel 124, which may include a spool for the guidewire 62. Therefore, in some embodiments, when the advancement wheel 124 is rotated, the rotation may cause the guidewire 62 to be advanced or retracted within the guidewire channel 116 depending on the direction in which the advancement wheel 124 is rotated.

As illustrated in FIG. 2D, in some embodiments, the rotary element 107 may include a spool 118 and the advancement wheel 124, both of which may be configured to rotate within the compartment 114. In some embodiments, the spool 118 may be positioned adjacent to the advancement wheel 124 (i.e., towards the guidewire channel 116 relative to the advancement wheel 124). In some embodiments, the advancement wheel 124 may be positioned to extend partially out from the compartment 114 to thereby enable the user to use his or her thumb or finger to rotate the advancement wheel 124. In some embodiments, the spool 118 may include a gear 119 having teeth. Likewise, in some embodiments, the advancement wheel 124 may include teeth and may therefore function as a gear. In some embodiments, the teeth of the advancement wheel 124 may interface with the teeth of the gear 119 so that the spool 118 is rotated when the advancement wheel 124 is rotated.

In some embodiments, the spool 118 and the advancement wheel 124 may include axles 120 and 121, respectively, by which these components are positioned within the compartment 114 and around which these components rotate. In some embodiments, the spool 118 may include a spool drum 122 around which the guidewire 62 may be wound. Therefore, when the spool 118 is rotated, the rotation may cause the guidewire 62 to be advanced or retracted within the guidewire channel 116 depending on the direction in which the advancement wheel 124 is rotated. In some embodiments, the advancement wheel 124 may include or correspond to any other advancement wheel described in further detail in U.S. patent application Ser. No. 17/709,935, filed Mar. 31, 2022, entitled “INSTRUMENT DELIVERY DEVICES, SYSTEMS, AND METHODS,” which is hereby incorporated by reference in its entirety.

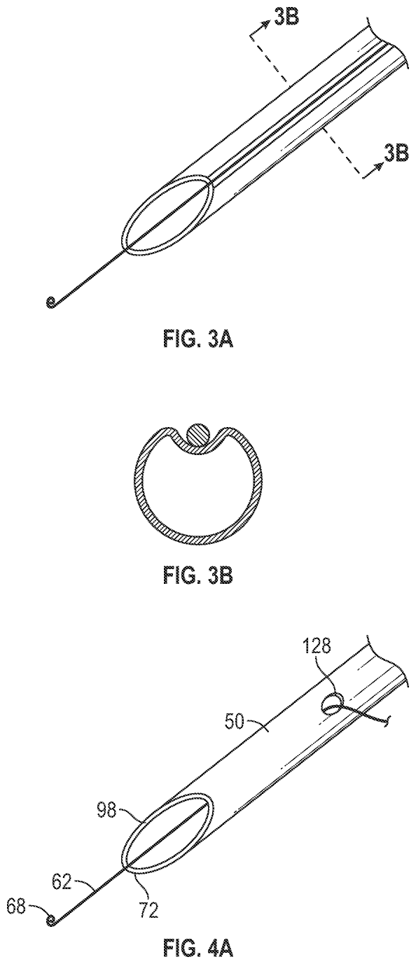

Referring now to FIGS. 3A-3B, in some embodiments, the introducer needle 50 may include an external groove 126. In some embodiments, the guidewire 62 may be disposed within the external groove 126 and may be configured to move through the external groove 126 to advance distally and/or be withdrawn proximally. In these and other embodiments, the guidewire advancement device 48 of FIGS. 1A-1C or the guidewire advancement device 106 of FIGS. 2A-2D may be used with the introducer needle 50 to advance and/or withdraw the guidewire 62 through the external groove 126. In some embodiments, the external groove 126 may extend along an entire length of the introducer needle 50, from the sharp distal tip 72 to the proximal end 60, which may facilitate advancement of the guidewire 62 along the entire length of the introducer needle 50. In some embodiments, a particular guidewire advancement device may be coupled to the proximal end 16 of the catheter adapter 12, and the guidewire 62 may be inserted straight through the catheter adapter 12 and the catheter 19 within the external groove 126.

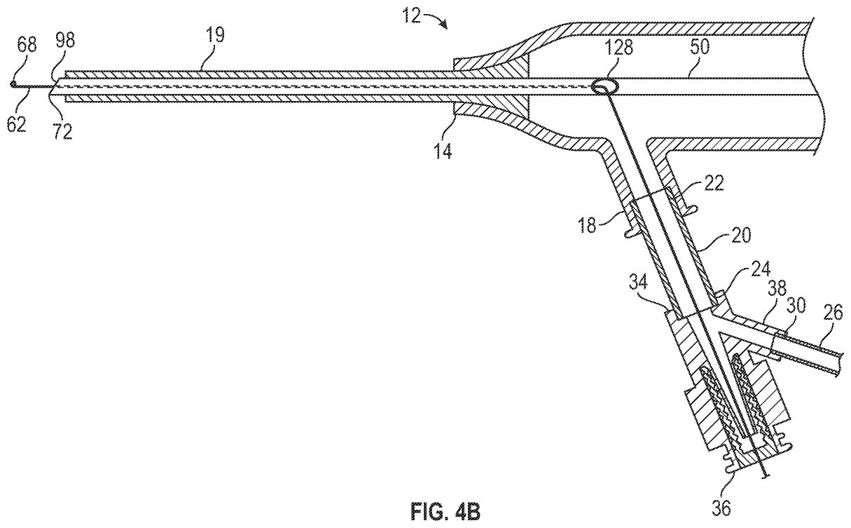

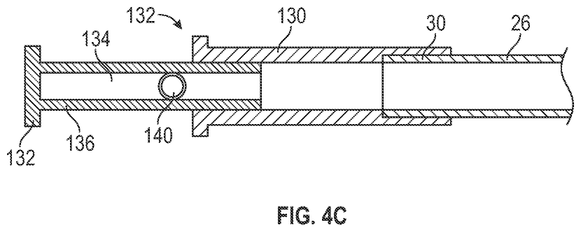

Referring now to FIGS. 4A-4B, in some embodiments, the introducer needle 50 may include an aperture 128 disposed between the sharp distal tip 72 and the proximal end 60 of the introducer needle 50. In some embodiments, the guidewire 62 may be configured to extend through the extension tube 20, the side port 18, and the aperture 128 and out the sharp distal tip 72 of the introducer needle 50. In these and other embodiments, the guidewire advancement device 48 of FIGS. 1A-1C or the guidewire advancement device 106 of FIGS. 2A-2D may be coupled to the proximal port 36 to advance and/or withdraw the guidewire 62 through the aperture 128 and the catheter 19.

Referring now to FIG. 4C, in some embodiments, the proximal end 30 of the second extension tube 26 may be integrated with an adapter 130, which may reduce a risk of fluid exposure to the user. In further detail, in some embodiments, the proximal end 30 of the second extension tube 26 may be permanently or non-removably coupled to the adapter 130, such as, for example, via adhesive, bonding, a non-luer coupling, or another suitable permanent or non-removable coupling. In some embodiments, the adapter 130 may be coupled to a needleless connector, which may reduce a risk of bacterial contamination. In some embodiments, a proximal end 132 of the adapter 130 may include a single port or a dual port.

In some embodiments, the catheter system 10 may include a flash chamber 134, which may be disposed within a plug 136 coupled to the adapter 130 to facilitate blood flashback visualization. In some embodiments, a proximal end 138 of the flash chamber 134 may be closed, and the flash chamber 134 may include an air vent 140 distal to the proximal end 138 of the flash chamber 134. In some embodiments, the air vent 140 may be configured to pass air and not blood. In some embodiments, the air vent 140 may be disposed towards a distal end of the flash chamber 134, which may maintain some unvented air in the flash chamber 134 to provide arterial pulse visualization, thereby providing sustained visual confirmation of proper arterial access.

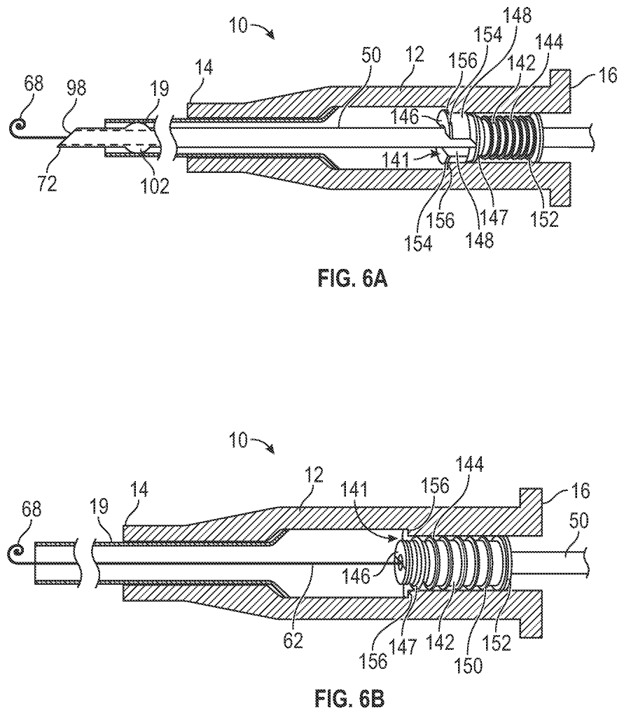

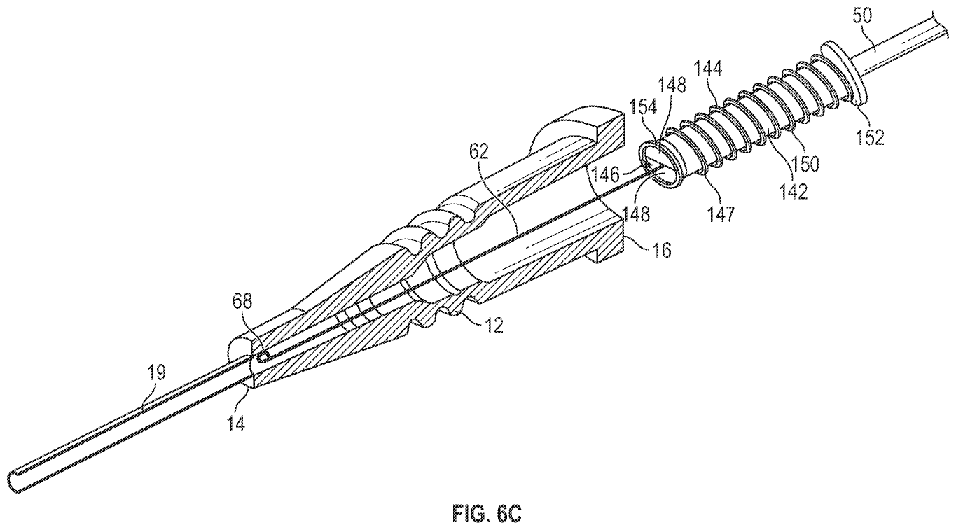

Referring now to FIGS. 6A-6C, the needle safety element may include an introducer needle capture mechanism 141, which may allow an introducer needle 50 to be moved from an unshielded position (see FIG. 6A, for example) to a shielded position (see FIG. 6B, for example) in which the introducer needle 50 is prevented from moving distally out of the capture mechanism 141. In some embodiments, the introducer needle 50 may be bi-directionally trapped and also prevented from moving proximally out of the capture mechanism 141. As used herein, the term “unshielded” may refer to circumstances in which the sharp distal tip of the introducer needle 50 is exposed from an inner housing of the capture mechanism 141. Conversely, the term “shielded” may refer to circumstances in which the sharp distal tip 72 of the introducer needle 50 is covered, shielded, or otherwise protected by the inner housing of the capture mechanism 141. In some embodiments, because the capture mechanism 141 allows the introducer needle 50 to be locked in the shielded position, the capture mechanism 141 may prevent unintended sticking and/or blood exposure.

In some embodiments, the introducer needle capture mechanism 141 may include an introducer needle 50 (e.g., introducer needle 50), a needle bump 102, an inner housing 142, and an outer housing 144. Additionally, in some embodiments, the capture mechanism 141 may be selectively coupled with a catheter adapter 12. In some embodiments, when the introducer needle 50 is exposed from the inner housing 142 in the unshielded position, the introducer needle 50 may extend axially through the inner housing 142 and the outer housing 144 so that the sharp distal tip 72 extends past a distal end 146 of the inner housing 142 and a distal end 147 of the outer housing 144. Additionally, in the unshielded position, a distal portion of the introducer needle 50 may extend into a catheter 19, and the outer housing 144 may be coupled to the catheter adapter 12.

In some embodiments, the inner housing 142 may be split longitudinally to form multiple arms 148. The inner housing 142 may include any number of arms, such as, for example, two, three, or four. In some embodiments, the arms 148 may extend distally from a proximal portion, such as a proximal end, of the inner housing 142. In some embodiments, when the introducer needle 50 is exposed from the distal end 146 of the inner housing 142, the introducer needle 50 may be configured to bias the arms 148 apart. For example, in some embodiments, when the introducer needle 50 is exposed from the distal end 146 of the inner housing 142 in the unshielded position, the arms 148 of the inner housing 142 may be resiliently splayed radially outward by the introducer needle 50, as illustrated, for example, in FIG. 6A.

In some embodiments, the inner housing 142 may be constructed of any suitable material or materials, such as, for example, a metal, a metal alloy, a ceramic, a plastic, a polymer, etc. Advantageously, a rigid plastic may allow the sharp distal tip 72 to embed in an inner surface of the inner housing 142 when in the shielded position. In some embodiments, the inner housing 142 may be a single piece. In some embodiments, the inner housing 142 may include multiple pieces that may be coupled together in any number of ways, such as, for example, threading, fitting, snapping, connecting, attaching, fastening, clipping, hooking, or any other suitable means of coupling.

In some embodiments, when the introducer needle 50 is retracted proximally within the inner housing 142 to the shielded position, the outer housing 144 may be configured to move from a compressed state, illustrated in FIG. 6A, to a decompressed state illustrated in FIG. 6B. In some embodiments, when the outer housing 144 is in the decompressed state, the outer housing 144 may bias the arms 148 of the inner housing 142 together, which may securely seal or close the distal end 146 of the inner housing 142 and prevent the introducer needle 50 from exiting the distal end 146 of the inner housing 142.

In some embodiments, the outer housing 144 may axially slide or expand over at least a portion of the inner housing 142 when the outer housing 144 decompresses or moves from the compressed state, illustrated, for example, in FIG. 2A, to the decompressed state, illustrated, for example, in FIG. 2B. The portion of the inner housing 142 over which the outer housing 144 slides or expands may be any suitable size or have any suitable shape. For instance, the portion of the inner housing 142 may be substantially cylindrical, cuboidal, tubular, etc. A restoring force in the distal direction may cause the outer housing 144 to axially slide or expand when the outer housing 144 moves from the compressed state to the decompressed state.

In some embodiments, the outer housing 144 may include a compressible portion 150, which may be disposed between and/or coupled with a distal end 147 of the outer housing 144 and a proximal end 152 of the outer housing 144. In some embodiments, the compressible portion 150 of the outer housing 144 may be compressed when the outer housing 144 is in the compressed state and decompressed when the outer housing 144 is in the decompressed state. In some embodiments, the compressible portion 150 may be compressible along an axis aligned with the introducer needle 50. In some embodiments, the compressible portion 150 may include a spring, an elastomer, or another compressible member. In some embodiments, the compressible portion 150 may be cylindrical. In some embodiments, the spring may be cylindrical and/or coiled. In some embodiments, the compressible portion 150 may be baffled or accordion-shaped. In these and other embodiments, the compressible portion 150 may include a tube or sleeve, which may be constructed of an elastomer, silicone, a liquid silicone rubber material, or another suitable material. In some embodiments, the compressible portion 150 may enclose the inner housing 142, which may act as a redundant mechanism to ensure any residual blood on the introducer needle 50 stays within the introducer needle capture mechanism 141.

In some embodiments, a proximal end 152 of the outer housing 144 may be directly coupled with the proximal end of the inner housing 142, and the distal end 147 of the outer housing 144 may move distally away from the proximal end of the outer housing 144 and the proximal end of the inner housing 142 when the outer housing 144 moves to the decompressed state. In some embodiments, movement of the outer housing 144 to the decompressed state may cause the distal end 147 of the outer housing 144, which may be radially rigid and/or coupled with the compressible portion 150, to slide or move distally along the inner housing 142.

In some embodiments, an inner surface of the catheter adapter 12 may be configured to secure the inner housing 142 within the catheter adapter 12 when the introducer needle 50 is exposed from the inner housing 142. In some embodiments, the inner surface of the catheter adapter 12 may be configured to separate from the inner housing 142 to release the inner housing 142 from the catheter adapter when the introducer needle 50 is retracted proximally within the inner housing 142, as illustrated in FIG. 6B. In further detail, in some embodiments, an outer surface of the inner housing may include one or more interlock components 154 that may interact with one or more interlock surfaces 156 formed in the inner surface of the catheter adapter 12. In some embodiments, the interlock components 154 may be biased against a corresponding interlock surface 156 of the catheter adapter 12 when the introducer needle 50 is exposed from the distal end 146 of the inner housing 142 in the unshielded position. In some embodiments, contraction of the inner housing 142 radially inward, in response to retraction of the introducer needle 50 proximally within the inner housing 142, may cause the one or more interlock components 154 to be separated from the one or more interlock surfaces 156, which may allow removal of the introducer needle capture mechanism 141 from the catheter adapter 12, the sharp distal tip 72 of the introducer needle 50 being shielded inside inner housing 142. Accordingly, unintentional needle sticks may be prevented, and the introducer needle capture mechanism 141 may be safely disposed.

In some embodiments, when the introducer needle 50 is retracted proximally within the inner housing 142 to the shielded position, the guidewire 62 may not be retracted and may remain proud of the introducer needle 50, as illustrated, for example, in FIG. 6B. In some embodiments, when the outer housing 144 is in the decompressed state, the outer housing 144 may bias the arms 148 of the inner housing 142 together, which may securely seal or close the distal end 146 of the inner housing 142 around the guidewire 62. In some embodiments, when the outer housing 144 is in the decompressed state, the outer housing 144 may bias the arms 148 of the inner housing 142 together and the arms 148 may pinch the guidewire 62, which may enhance the seal around the guidewire 62 and/or facilitate removal of the guidewire 62 with the introducer needle 50. In some embodiments, after the introducer needle is shielded in the inner housing 142, the guidewire 62 and the introducer needle 50 may be withdrawn proximally together and removed from the catheter system 10. In some embodiments, further details of the needle capture mechanism 141 may be further described in U.S. Pat. No. 10,507,281, filed Mar. 16, 2017, entitled “CANNULA CAPTURE MECHANISM,” which is hereby incorporated by reference in its entirety.

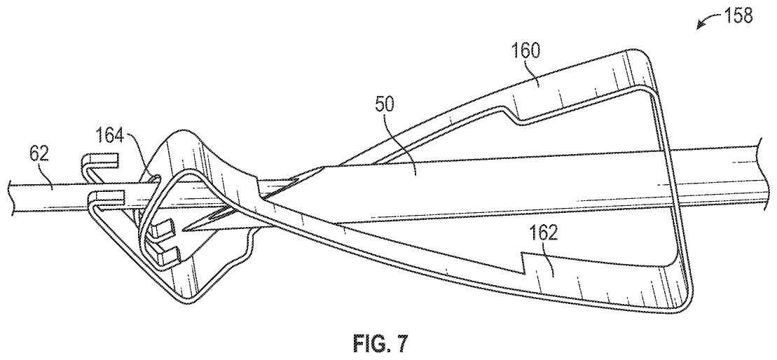

Referring now to FIG. 7, a crossing-arm clip 158, another type of spring clip, is illustrated, according to some embodiments. In some embodiments, the V-clip needle shield 46 of FIGS. 1B-1C of FIGS. 2B-2C may be replaced with the crossing-arm clip 158 or another suitable spring clip or needle safety element. In these embodiments, the crossing-arm clip 158 may be disposed within the safety housing 44. In other embodiments, the crossing-arm clip 158 may be disposed within a catheter adapter, such as, for example, the catheter adapter 12 of FIG. 1 of another suitable catheter adapter. In some embodiments, the crossing-arm clip 158 may include a resilient first arm 160 and a resilient second arm 162 configured to cross each other. In some embodiments, proximal ends of the first arm 160 and the second arm 162 may join at a proximal wall, which may include an opening sized and configured to slidably receive a proximal end of the introducer needle 50 as the introducer needle 50 is advanced in a distal direction. In some embodiments, a needle feature or bump (not illustrated) may prevent the sharp distal tip 72 of the introducer needle 50 from being withdrawn proximally through the opening as a diameter of the needle feature or bump may be larger than a diameter of the opening.

In some embodiments, distal ends of the first arm 160 and the second arm 162 may be curved and/or include a lip. In some embodiments, when the introducer needle 50 is in the insertion position, ready for insertion into the patient, the introducer needle 50 may be disposed between the distal end of the first arm 160 and the distal end of the second arm 162, biasing the first and second arms 160,162 outwardly. In some embodiments, when the first and second arms 160,162 are biased outwardly they may engage an inner wall of the catheter adapter or an inner wall of the safety housing 44, securing the crossing-arm clip 158 within the catheter adapter or the safety housing 44. In response to withdrawal of the introducer needle 50 from the patient and movement of the sharp distal tip 72 proximal to the distal ends of the first and second arms 160,162, the distal ends of the first and second arms 160, 162 may move closer to each other and/or overlap, which may release the crossing-arm clip 158 from the catheter adapter or the safety housing 44 and prevent the introducer needle 50 from moving in a distal direction beyond the crossing-arm clip 158, as illustrated in FIG. 7, for example.

In some embodiments, the crossing-arm clip 158 may not be modified for the guidewire 62 and/or may include a clip described further in U.S. Pat. No. 7,972,313, filed Nov. 22, 2006, entitled “SPRING CLIP SAFETY IV CATHETER,” which is hereby incorporated by reference in its entirety. In some embodiments, the distal end of the first arm 160 and/or the distal end of the second arm 162 may include an aperture 164 sized and configured to receive the guidewire 62. In some embodiments, a diameter of the aperture may be less than the diameter of the introducer needle 50, which may prevent the introducer needle 50 from moving distally through the aperture in the shielded position. In some embodiments, the crossing-arm clip 158 may be further described in U.S. Pat. No. 11,291,803, filed Jan. 3, 2019, entitled “CATHETER SYSTEM WITH GUIDEWIRE ADVANCEMENT ELEMENT,” which is hereby incorporated by reference in its entirety.

Referring now to FIGS. 8A-8B, in some embodiments, the guidewire advancement device 165 may include a rack 166 and a pinion 168. In some embodiments, the advancement tab 66 may include the rack 166. In some embodiments, the advancement tab 66 may be similar or identical to the advancement tab 66 of FIGS. 1-2 in terms of one or more features and/or operation with respect to the slot 64. In some embodiments, the guidewire advancement device 165 may be similar or identical to the guidewire advancement device 48 and/or the guidewire advancement device 106 in terms of one or more features and/or operation. In some embodiments, the pinion 168 may include a spool for the guidewire 62. Therefore, when the rack 166 is moved to rotate the pinion 168, the rotation may cause the guidewire 62 to be advanced or retracted depending on the direction in which rack 166 is moved and the pinion 168 is rotated.

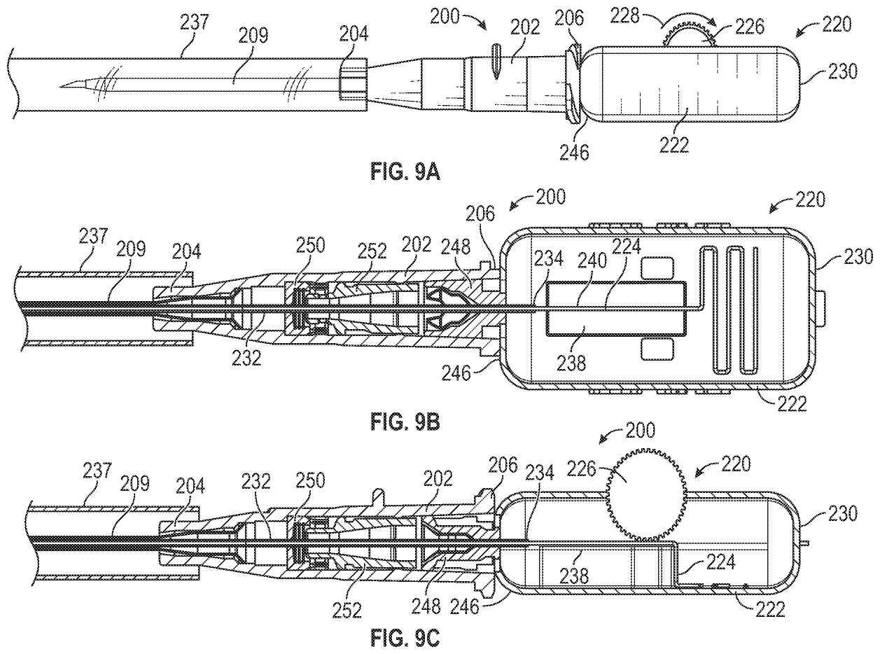

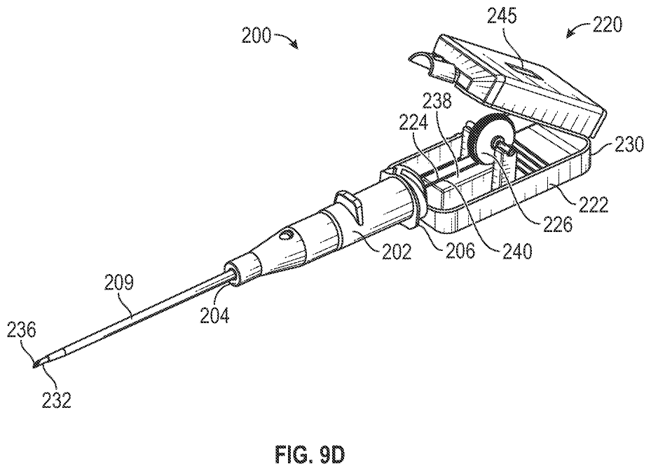

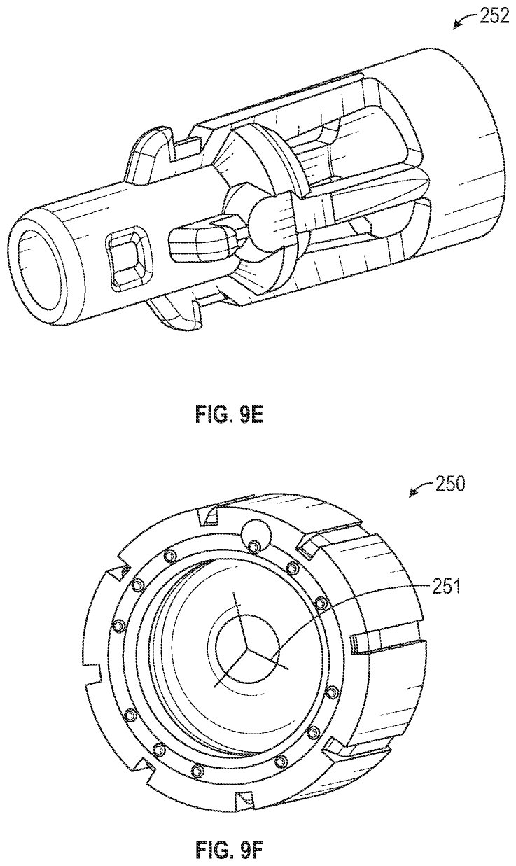

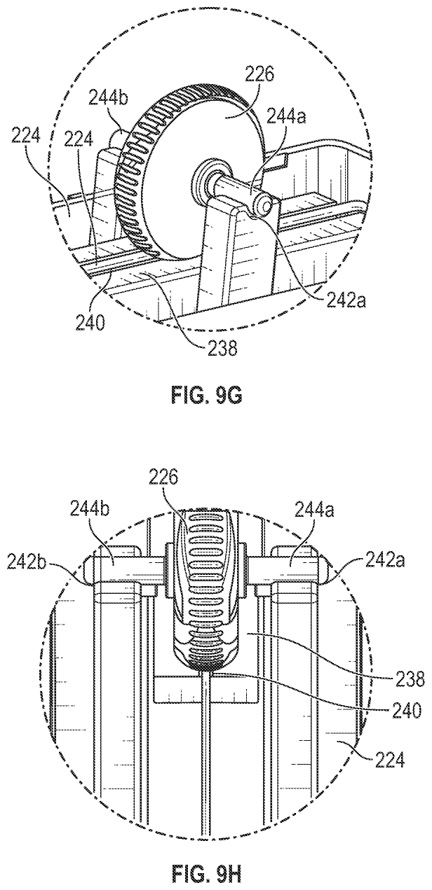

Referring now to FIGS. 9A-9H, a catheter system 200 is illustrated, according to some embodiments. In some embodiments, the catheter system 200 may be similar or identical to FIGS. 1-8 in terms of one or more components and/or operation. In some embodiments, the catheter system 200 may include a catheter adapter 202, which may include a distal end 204, a proximal end 206, a lumen extending through the distal end 204 of the catheter adapter 202 and the proximal end 206 of the catheter adapter 202. In some embodiments, the catheter system 200 may include a catheter 209 extending from the distal end 204 of the catheter adapter 202.

In some embodiments, the catheter system 200 may include a guidewire advancement device 220 coupled to the catheter adapter 202. In some embodiments, the guidewire advancement device 220 may include a casing 222, a guidewire 224 at least partially disposed within the casing 222, and a roller wheel element 226 extending through the casing 222 and contacting the guidewire 224. In some embodiments, in response to rotation of the roller wheel element 226 in a direction 228 that is proximal or towards a proximal end 230 of the casing 222, the guidewire 224 may be configured to advance distally. In these embodiments, the roller wheel element 226 may slightly pinch or compress the guidewire 224 as the roller wheel element 226 rotates or turns on a central axis, which may facilitate advancement of the guidewire 224.

In some embodiments, the catheter system 200 may include an introducer needle 232, which may include a proximal end 234 and a sharp distal tip 236. In some embodiments, a removable cover 237 may be disposed over the sharp distal tip 236 to prevent accidental needle stick. In some embodiments, the proximal end 234 of the introducer needle 232 may be secured within the guidewire advancement device 220. In some embodiments, the introducer needle 232 may extend through the catheter 219, and the guidewire 224 may be configured to move through the introducer needle 232 when the guidewire 224 is advanced distally.

In some embodiments, the catheter system 200 may include a platform 238 disposed within the casing 222 and below the roller wheel element 226. In some embodiments, the platform 238 may contact and support the guidewire 224. In some embodiments, the platform 238 may include a linear groove 240. In some embodiments, the guidewire 224 may be disposed within the linear groove 240, which may facilitate guidance of the guidewire 224 during distal advancement of the guidewire 224. In some embodiments, the platform 238 may be generally planar.

In some embodiments, the catheter system 200 may include opposing roller wheel support grooves 242a, 242b disposed within the casing 222. In some embodiments, the roller wheel element 226 may include opposing pins 244a, 244b fixed with respect to a circular body of the roller wheel element 226. In some embodiments, the opposing pins 244a, 244b may be configured to rotate within the opposing roller wheel support grooves 242a, 242b. In other embodiments, the circular body may rotate on an axle. In some embodiments, the circular body may extend through a slot 245 within the casing 222 and may be configured for easy rotation by a finger of a user.

In some embodiments, a proximal end of the guidewire 224 may be looped back on itself, coiled, or otherwise organized to facilitate distal movement and prevent tangling. In some embodiments, the proximal end of the guidewire 224 may be disposed on a floor of the casing 222 below the platform 238. In some embodiments, in response to rotation of the roller wheel element 226 in the direction 228 that is proximal or towards a proximal end 230 of the casing 222, the guidewire 224 may be configured to advance distally, including through the introducer needle 232 and the catheter 209 and into vasculature of a patient.

In some embodiments, a distal end 246 of the casing 222 may include a male luer fitting 248, which may facilitate coupling to the catheter adapter 202 and/or a safety housing, such as, for example, the safety housing 44 of FIGS. 1-2. In some embodiments, the distal end 246 of the casing 222 may include another type of suitable connector. In some embodiments, the safety housing may include a needle safety element, such as, for example, the V-clip needle shield 46 as previously described or another suitable needle safety element.

In some embodiments, the catheter system 200 may include a septum 250 and a septum actuator 252 disposed within the lumen of the catheter adapter 202. In some embodiments, the septum actuator 252 may be used to open the septum 250 in response to engagement of an external connector or insertion of an external connector into the proximal end 206 such that the septum actuator 252 is moved distally through the septum 250, which may include a slit 251. In some embodiments, in response to removal of the external connector from the proximal end 206, the septum actuator 252 may return to a proximal position within the lumen, and the septum 250 may close. Thus, in some embodiments, the septum 250 may help stop blood flow through the catheter adapter 202 when the catheter adapter 202 is not engaged with the external connector. In some embodiments, a fluid path through the catheter adapter 202 may be closed unless the external connector is engaged with the catheter adapter 202 such that the septum actuator 252 is moved distally and opens the septum 250.

In some embodiments, the user may remove the removable needle cover 237 and make sure the guidewire 224 is inside the introducer needle 232. In some embodiments, before insertion of the introducer needle 232 and the catheter 209 into the skin of the patient, tip adhesion may be broken. In some embodiments, after the introducer needle 232 and the catheter 209 is inserted through the skin and into the blood vessel, blood flashback may be observed by the clinician in a flashback chamber of the catheter system 200 or via a notch in the introducer needle 232, allowing blood to flow between the introducer needle 232 and the catheter 209. In some embodiments, after the clinician observes the blood flashback confirming entry of the introducer needle 232 into the blood vessel, the roller wheel element 226 may be rotated to move the guidewire 224 through the introducer needle 232.

In some embodiments, after the guidewire 224 is advanced through the introducer needle 232 and into the blood vessel, the catheter adapter 202 may be advanced distally to further move the catheter 209 into the blood vessel. In some embodiments, after the catheter 209 is moved further into the blood vessel, the casing 222 may be uncoupled from the catheter adapter 202 and slowly moved proximally by the user, withdrawing the introducer needle 232 and the guidewire 224 from the catheter adapter 202 and the catheter system 200. In some embodiments, the casing 222, introducer needle 232, and the guidewire 224 may all be discarded into an appropriate sharps container.

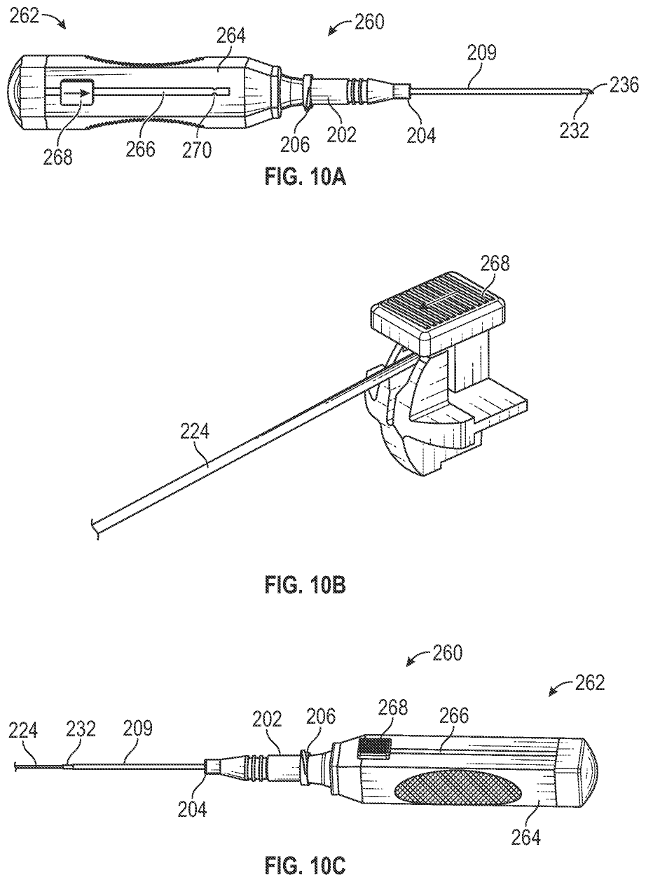

Referring now to FIGS. 10A-10C, a catheter system 260 is illustrated. In some embodiments, the catheter system 260 may be similar or identical to FIGS. 1-9 in terms of one or more components and/or operation. In some embodiments, the catheter system 260 may include a guidewire advancement device 262 coupled to the catheter adapter 202. In some embodiments, the guidewire advancement device 262 may include a casing 264, a slot 266 within the casing 264, the guidewire 224, and an advancement tab 268 extending through the slot 266 and configured to move linearly along the slot 266 to advance the guidewire 224 distally. In some embodiments, a shape of the advancement tab 268 may facilitate smooth, supported movement through a lumen of the casing 264. In some embodiments, the guidewire 224 may be coupled to and extend distally from the advancement tab 268. In some embodiments, the slot 266 may include a snap feature 270, which may engage with the advancement tab 268 to prevent the advancement tab 268 from moving proximally after advancement, reducing a risk of shearing the guidewire 224 on the introducer needle 232.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter assembly, comprising:
a catheter adapter, comprising a distal end, a proximal end, a lumen extending through the distal end of the catheter adapter and the proximal end of the catheter adapter, and a side port between the distal end of the catheter adapter and the proximal end of the catheter adapter and in fluid communication with the lumen;
a catheter extending from the distal end of the catheter adapter;
an extension tube comprising a distal end and a proximal end, wherein the distal end of the extension tube is integrated with the side port of the catheter adapter;
a needle assembly, comprising:
a needle safety element disposed within the safety housing;
a guidewire advancement device, comprising a distal end, a proximal end, and a guidewire; and
an introducer needle comprising a proximal end and a sharp distal tip, wherein the proximal end of the introducer needle is coupled to the guidewire advancement device, wherein the introducer needle extends through the catheter, wherein the needle safety element is configured to shield the sharp distal tip.

2. The catheter system of claim 1, wherein removal of the guidewire advancement device from the catheter system is configured to withdraw the guidewire and the introducer needle from the catheter system, wherein the guidewire is configured to remain proud of the introducer needle when the guidewire and the introducer needle are withdrawn from the catheter system.

3. The catheter system of claim 1, wherein the guidewire advancement device comprises a slot and an advancement tab moveable linearly along the slot, wherein the advancement tab is configured to advance the guidewire, wherein a longitudinal axis of the guidewire advancement device is aligned with a longitudinal axis of the catheter assembly.

4. The catheter system of claim 3, wherein the guidewire advancement device comprises a rack and pinion, wherein the advancement tab comprises the rack.

5. The catheter system of claim 1, wherein the guidewire advancement device comprises a rotary element configured to advance the guidewire.

6. The catheter system of claim 1, wherein the introducer needle comprises a lumen extending through the sharp distal tip and the proximal end, wherein the guidewire is disposed within the lumen and is configured to move through the proximal end of the introducer needle to advance.

7. The catheter system of claim 1, wherein the introducer needle comprises an external groove, wherein the guidewire is disposed within the external groove and configured to move through the external groove to advance distally.

8. The catheter system of claim 1, further comprising a safety housing, wherein the safety housing comprises a distal end and a proximal end, wherein the distal end of the safety housing is coupled to the proximal end of the catheter adapter, wherein the needle safety element comprises a V-clip needle shield, wherein the distal end of the guidewire advancement device is coupled to the proximal end of the safety housing, wherein the safety housing comprises an open space proximate and proximal to the V-clip needle shield, wherein the guidewire is configured to bend within the open space when the V-clip needle shield fires.

9. The catheter system of claim 8, wherein the introducer needle comprises a sharp distal tip, a proximal end, and a lumen extending through the sharp distal tip and the proximal end, wherein the sharp distal tip comprises a bevel facing upward, wherein the V-clip needle shield is configured to fire side to side.

10. The catheter system of claim 1, further comprising a safety housing, wherein the safety housing comprises a distal end and a proximal end, wherein the distal end of the safety housing is coupled to the proximal end of the catheter adapter, wherein the needle safety element comprises a V-clip needle shield, wherein the distal end of the guidewire advancement device is coupled to the proximal end of the safety housing, wherein the introducer needle comprises a sharp distal tip, a proximal end, and a lumen extending through the sharp distal tip and the proximal end, wherein the sharp distal tip comprises a bevel facing upward, wherein the V-clip needle shield is configured to fire in an upward or downward direction.

11. The catheter system of claim 1, wherein the extension tube is a first extension tube, further comprising:
an access connector, wherein the access connector comprises a distal port, a proximal port, and a side port between the distal port and the proximal port, wherein the distal port and the proximal port are aligned with a longitudinal axis of the access connector, wherein the side port is angled with respect to the longitudinal axis of the access connector, wherein the proximal end of the first extension tube is integrated with the distal port of the access connector; and
a second extension tube comprising a distal end and a proximal end, wherein the distal end of the second extension tube is integrated with the side port of the access connector, wherein the first extension tube is shorter than the second extension tube, wherein the longitudinal axis of the access connector, the first extension tube, and the side port are configured to align to form a straight path.

12. The catheter system of claim 11, further comprising an adapter coupled to the proximal end of the second extension tube, a plug coupled to the adapter, a flash chamber disposed within the plug, and an air vent distal to a proximal end of the flash chamber, wherein the proximal end of the second extension tube is integrated with the adapter, wherein the proximal end of the flash chamber is closed, wherein the air vent is configured to pass air and not blood such that the plug is configured to maintain unvented air in the proximal end of the flash chamber to facilitate arterial pulse visualization.

13. The catheter system of claim 11, further comprising a catheter advancement device coupled to the proximal port.

14. The catheter system of claim 11, wherein the introducer needle comprises a sharp distal tip, a proximal end, a lumen extending through the sharp distal tip and the proximal end, and an aperture disposed between the sharp distal tip and the proximal end, wherein the guidewire is configured to extend through the extension tube, the side port, and the aperture and out the sharp distal tip of the introducer needle.

15. The catheter system of claim 1, wherein the needle safety element comprises a crossing-arm clip.

16. A method, comprising:
inserting a catheter system into a blood vessel of a patient, wherein the catheter system comprises:
a catheter assembly, comprising:
a catheter adapter, comprising a distal end, a proximal end, a lumen extending through the distal end of the catheter adapter and the proximal end of the catheter adapter, a side port between the distal end of the catheter adapter and the proximal end of the catheter adapter and in fluid communication with the lumen;
a catheter extending from the distal end of the catheter adapter; and
an extension tube comprising a distal end and a proximal end,
wherein the distal end of the extension tube is integrated with the side port of the catheter adapter; and
a needle assembly, comprising:
a needle safety element;
a guidewire advancement device, comprising a distal end, a proximal end, and a guidewire; and
an introducer needle comprising a proximal end coupled to the guidewire advancement device, wherein the introducer needle extends through the catheter;
after inserting the catheter system into the blood vessel, advancing the guidewire distally;
after advancing the guidewire distally, removing the guidewire advancement device from the catheter system, wherein removal of the guidewire advancement device from the catheter system withdraws the guidewire and the introducer needle from the catheter system, wherein the guidewire remains proud of the introducer needle when the guidewire and the introducer needle are withdrawn from the catheter system.

17. The method of claim 16, wherein the extension tube is a first extension tube, wherein the catheter system further comprises:
an access connector, wherein the access connector comprises a distal port, a proximal port, and a side port between the distal port and the proximal port, wherein the distal port and the proximal port are aligned with a longitudinal axis of the access connector, wherein the side port is angled with respect to the longitudinal axis of the access connector, wherein the proximal end of the first extension tube is integrated with the distal port of the access connector; and
a second extension tube comprising a distal end and a proximal end, wherein the distal end of the second extension tube is integrated with the side port of the access connector, wherein the first extension tube is shorter than the second extension tube.

18. The method of claim 17, further comprising after removing the guidewire advancement device from the catheter system, wherein removal of the guidewire advancement device from the catheter system withdraws the guidewire and the introducer needle from the catheter system:
coupling a catheter advancement device to the proximal port and advancing a secondary catheter into the catheter assembly via the catheter advancement device; or
coupling a probe advancement device to the proximal port and advancing the probe advancement device into the catheter assembly via the probe advancement device.

19. The method of claim 16, wherein the needle safety element comprises a V-clip needle shield, a crossing-arm clip, or an introducer needle capture mechanism.

20. The method of claim 16, wherein the needle assembly further comprises a safety housing, wherein the safety housing comprises a distal end and a proximal end, wherein the distal end of the safety housing is coupled to the proximal end of the catheter adapter, wherein the needle safety element is disposed within the safety housing, wherein the distal end of the guidewire advancement device is coupled to the proximal end of the safety housing.

21. A catheter system, comprising:
a catheter adapter, comprising a distal end, a proximal end, a lumen extending through the distal end of the catheter adapter and the proximal end of the catheter adapter;
a catheter extending from the distal end of the catheter adapter;
a guidewire advancement device coupled to the catheter adapter, wherein the guidewire advancement device comprises a casing, a guidewire at least partially disposed within the casing, and a roller wheel element extending through the casing and contacting the guidewire, wherein in response to rotation of the roller wheel element in a proximal direction, the guidewire is configured to advance distally; and
an introducer needle comprising a proximal end and a sharp distal tip, wherein the proximal end is secured within the guidewire advancement device, wherein the introducer needle extends through the catheter.

22. The catheter system of claim 21, further comprising a platform disposed within the casing and below the roller wheel element, wherein the platform comprises a linear groove, wherein the guidewire is disposed within the linear groove.

23. The catheter system of claim 21, further comprising opposing roller wheel support groves disposed within the casing, wherein the roller wheel element comprises opposing pins configured to rotate within the opposing support grooves.

24. The catheter system of claim 21, wherein a distal end of the casing comprises a male luer fitting.

25. The catheter system of claim 21, further comprising a septum and a septum actuator disposed within the lumen of the catheter adapter.

26. A catheter system, comprising:
a catheter adapter, comprising a distal end, a proximal end, a lumen extending through the distal end of the catheter adapter and the proximal end of the catheter adapter;
a catheter extending from the distal end of the catheter adapter;
a guidewire advancement device coupled to the catheter adapter, wherein the guidewire advancement device comprises a casing, a slot within the casing, a guidewire, and an advancement tab extending through the slot and configured to move along the slot to advance the guidewire distally; and
an introducer needle comprising a proximal end and a sharp distal tip, wherein the proximal end is secured within the guidewire advancement device, wherein the introducer needle extends through the catheter.
